(19) United States
(12) Patent Application Publication
(10) Pub. No.: US 2006/0229681 A1
(43) Pub. Date: Oct. 12, 2006

(54) Implantable system for the treatment of atrial fibrillation

(76) Inventor: Robert E. Fischell, Dayton, MD (US)

Correspondence Address: Robert E. Fischell, 14600 Vibornom Drive, Dayton, MD, 21036, US

(21) Appl. No.: 11/103,141

(22) Filed: Apr. 11, 2005

(51) Int. Cl. A61N 1/39

(52) U.S. Cl. 607/5; 607/14

(57) ABSTRACT

Disclosed is a closed-loop system that utilizes an implanted responsive tissue stimulator connected electrically to sensing and stimulation electrodes that can detect a precursor of atrial fibrillation or the atrial fibrillation signal itself and then apply electrical stimulation onto some of the patient's tissue such that the atrial fibrillation episode is prevented or terminated. One region for stimulation is at or near the vagal nerves in the neck so as to increase the patient's parasympathetic activity which is known to decrease a tendency for atrial fibrillation. Another region that could be stimulated to decrease the probability of the onset of atrial fibrillation is the region of the left atrium near or in the ostium of the pulmonary veins. Data on the occurrence of atrial fibrillation is recorded in a digital memory so that the patient's doctor can read out these data to optimize the treatment of atrial fibrillation.

This invention is in the field of methods and devices for the treatment of atrial fibrillation.

### BACKGROUND OF THE INVENTION

Atrial fibrillation is a disorder of the heart that (in the year 3005) affects more than 2 million Americans and a comparable number of patients outside the USA. There have been many treatments for this disorder including surgery and percutaneous catheter treatments that require mapping of the electrical signals within the heart and then ablative destruction of the region of the pulmonary vein that is in close proximity to the left atrium. These treatments often have serious side effects and are not always effective for the treatment of atrial fibrillation.

Their have been many studies that have shown that there are certain electrical signals in the heart that are precursors of episodes of atrial fibrillation. Unfortunately, each patient can have a somewhat different precursor electrical signal as compared to another patient. Therefore, there is no data to date that all patients have the same precursor signal that comes before an episode of atrial fibrillation. There have also been several studies that have shown that electrical stimulation of the vagal nerve in the neck can increase parasympathetic tone of the nervous system that can decrease the propensity for an episode of atrial fibrillation. However, no data exists on combining the detection of electrical precursors of atrial fibrillation with the application of electrical stimulation of the vagus nerve to prevent the occurrence of atrial fibrillation.

### SUMMARY OF THE INVENTION

The present invention is an implantable system for the treatment of atrial fibrillation. The operation of the system is based upon the concept that each patient who has atrial fibrillation will also have a specific electrical signal precursor of the atrial fibrillation that is detectable by one or more intracardiac electrodes. If there is no detectable precursor of the patient's atrial fibrillation, then there is at least the ability to detect the early onset of an episode of atrial fibrillation and then apply a responsive electrical treatment. Before any such system is implanted, a Halter monitor or an implanted arrhythmia detector (such as the Reveal device of Medtronic, Inc.) would make an exact determination as to the type of precursor signal that a specific patient has prior to an episode of atrial fibrillation. If the patient exhibits such a precursor electrical signal, then the implanted system as described herein could be used to sense such a precursor and stimulate some tissue of that patient to prevent the occurrence of atrial fibrillation. If there is no precursor signal, then the start of the episode of atrial fibrillation can be used to trigger a responsive electrical treatment to turn off the atrial fibrillation. The present invention is utilizes upon the concept that, although the precursor signal may differ from one patient to another, the precursor signal for a particular patient would be repeatable for that patient.

An important aspect of the present invention is the selection of the region to be stimulated. One region can be the vagus nerve in the neck. Prior studies have shown that such electrical vagal stimulation can increase the parasympathetic tone of the nervous system which is known to decrease the probability of the occurrence of atrial fibrillation. Such stimulation could be applied immediately upon the detection of a precursor signal of atrial fibrillation or the early onset of the atrial fibrillation. It would probably be reasonable to expect that, once applied, such an electrical stimulation signal would be maintained for several seconds or even hours before it is turned off. Although it is possible to stimulate both the left and the right vagus nerves to increase parasympathetic tone, the vagal stimulation of the left vagus nerve is the preferred modality for the present invention.

Another region that could be stimulated to decrease the probability of the onset of atrial fibrillation is the region of the left atrium near or in the ostium of the pulmonary veins. Since there are four pulmonary veins that enter the left atrium, one might determine by electrical mapping of the electrical signals in the heart that cause atrial fibrillation, which one or more of the pulmonary veins is the source of the aberrant electrical signal that results in the occurrence of atrial fibrillation. The stimulation electrode might even be connected to a metal stent placed at the ostium of one or more pulmonary vein in order to most efficiently disrupt the aberrant electrical signal that causes the heart to have atrial fibrillation.

In any case, the present invention utilizes a responsive tissue stimulator connected electrically to sensing and stimulation electrodes whereby the precursor of atrial fibrillation or the atrial fibrillation signal itself is sensed and an electrical stimulation signal is applied onto some of the patient's tissue such that the atrial fibrillation episode is prevented or terminated. The responsive tissue stimulator also must have the typical electrical and electronic components that include a battery, d-c to d-c converter, signal amplifiers, analog-to-digital converters, digital memory, command receivers for receiving signals to program the implant and a telemetry system to transmit to external equipment the state of the implanted responsive tissue stimulator and the contents of the memory. This type of responsive electrical stimulation system is described in some detail in many US patents including U.S. Pat. No. 6,016,449. What is truly unique about the present invention is that there is no prior art relative to a closed-loop system for sensing a precursor or start of atrial fibrillation in a specific patient, having an implanted responsive tissue stimulator recognize that specific signal pattern and then applying electrical stimulation to some of the patient's tissue to prevent the occurrence of atrial fibrillation.

Thus one object of the present invention is to have a responsive tissue stimulation system implanted within a human subject to detect a precursor or early onset electrical signal of atrial fibrillation for that specific patient and then apply an electrical stimulation signal onto some of the patient's tissue to prevent the occurrence of that episode of atrial fibrillation.

Another object of this invention is to maintain that responsive stimulation for a preset but programmable time period, which time period is sufficient to decrease the occurrence rate of atrial fibrillation.

Still another object of this invention is to make the sensing and stimulation parameters of the implanted electrical stimulation system programmable by means of a command rf signal from external equipment.

Throughout this specification, whenever a precursor of atrial fibrillation is described it also includes sensing of the early onset electrical signal of the atrial fibrillation itself.

These and other objects and advantages of this invention will become obvious to a person of ordinary skill in this art upon reading the detailed description of this invention including the associated drawings as presented herein.

### BRIEF DESCRIPTION OF THE DRAWINGS

FIG. 1 illustrates the responsive tissue stimulator system including the responsive tissue stimulator connected by wire leads to the sensing and stimulation electrodes and the external equipment that communicates with the responsive tissue stimulator.

FIG. 2 is a block diagram of the responsive tissue stimulator system including the implanted and external equipment portions of the system.

### DETAILED DESCRIPTION OF THE INVENTION

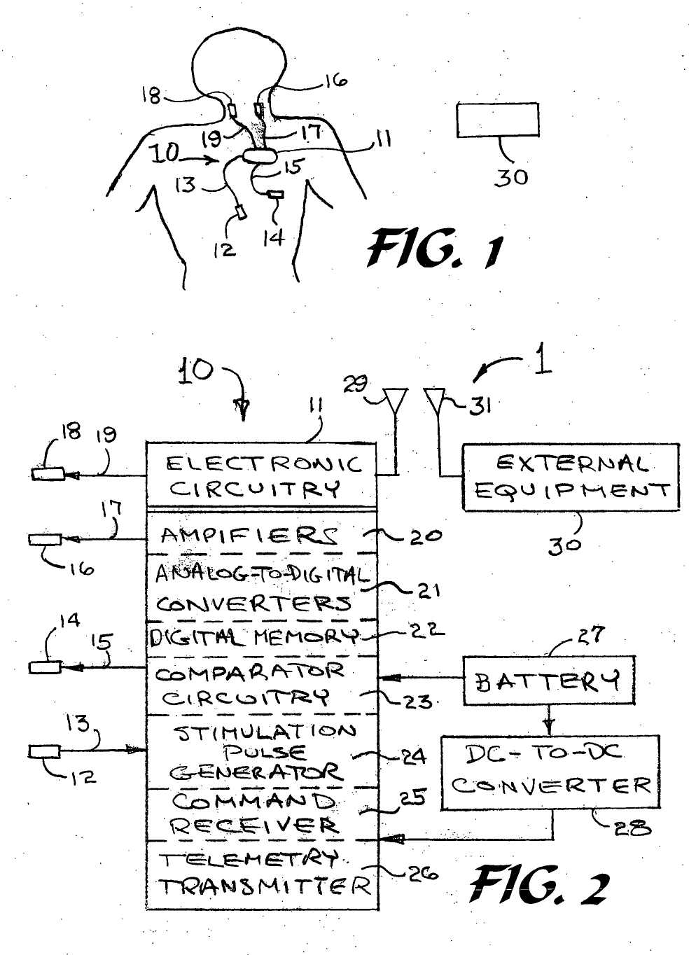

FIG. 1 illustrates the implanted and external equipment portions of the responsive tissue stimulator system 1. This system 1 consists of an implanted portion 10 and external equipment 30. The implanted portion 10 consists of a responsive tissue stimulator 11 that is electrically connected to the sensing electrode 12 by means of the wire lead 13, the stimulation electrode 14 connected via the wire 15, the stimulation electrode 16 connected via the wire 17 and the stimulation electrode 18 connected to the responsive tissue stimulator 11 by the wire 19. The electrical circuitry of the responsive tissue stimulator 10 and the external equipment 30 are both described in more detail within the description of FIG. 2 below.

The implanted portion 10 of the responsive tissue stimulator system 1 utilizes its responsive tissue stimulator 11 to sense the electrical signal from any sensing electrode placed within the patient's body. One such sensing electrode 12 might advantageously be located in or near the apex of the right ventricle as is the current practice for present day pacemakers and implantable cardioverter defibrillators (ICDs). Wherever the precursor signal for atrial fibrillation is most prevalent is where such a sensing electrode 12 should be placed. One such location could be within the left or right atrium.

The stimulation electrodes 14, 16 and 18 should be placed wherever electrical stimulation can cause a decrease in the rate of having atrial fibrillation episodes. Examples of such locations are the left vagus nerve in the region of the neck or possibly simultaneous stimulation of both the left and right vagus nerves in the neck or elsewhere in the body or possibly in or near the ostia of the pulmonary veins that feed into the left atrium. It is well known that causing tissue damage to one or more pulmonary vein in close proximity to the left atrium can result in a decrease in the occurrence of episodes of atrial fibrillation. This is because such tissue damage interrupts aberrant electrical signals that cause the atrial fibrillation. The present invention anticipates that, after mapping out the electrical signals from within the heart that are the cause of atrial fibrillation, one could apply a rather high intensity electrical signal near or within the ostia of one or more of the pulmonary veins in response to the detection by some sensing electrode that a precursor signal of atrial fibrillation has been detected. By making such a system responsive rather than constantly applied, battery life of the implanted responsive tissue stimulator 11 would be greatly extended. In any case, after a precursor of an episode of atrial fibrillation is detected by some sensing electrode (e.g., the electrode 12) the responsive tissue stimulator 11 can apply an electrical stimulation to some of the patient's tissue(s) which application can decrease the extent or severity of atrial fibrillation. Such a responsive stimulation signal could last from as short as a fraction of a second to as long as several hours depending on the actual experience with a specific patient. As episodes of atrial fibrillation become more controlled and less frequent, the time duration for keeping the electrical stimulation applied can undoubtedly be decreased.

FIG. 2 is a block diagram of the responsive tissue stimulator system 1 that includes the implanted portion 10 and the external equipment 30. The implanted portion 10 consists of the responsive tissue stimulator 11 and several electrodes and electrical wires that connect the electrodes to the responsive tissue stimulator 11. There could be a multiplicity of sensing electrodes such as the sensing electrode 12 connected to the responsive tissue stimulator 11 via the wire lead 13. The location of any sensing electrode (such as the electrode 12) would be where the precursor signal for atrial fibrillation is most easily detected. One such location would be at the apex of the right ventricle. One or more stimulation electrodes should be located where they can best be used to prevent the occurrence of the episode of atrial fibrillation that follows the precursor signal in a particular patient for that episode of atrial fibrillation. It is understood that different patients will have a different precursor signal, but the same patient will have a repeatable precursor signal. In FIG. 2, three different stimulations electrodes 14, 16 and 18 are shown. Stimulation could usefully be applied to the vagus nerve to increase the parasympathetic tone of the vagus nerve thus decreasing the propensity for atrial fibrillation. Another approach would be to stimulate the left atrium at or near the ostium of one or several pulmonary veins. The electrode could be a metal stent that is located in that one or more of the pulmonary veins through which the mapping of the heart's electrical signals has shown to be associated with the occurrence of atrial fibrillation. One purpose of such a stent would be to prevent stenosis of the pulmonary vein which is known to be a very serious side effect of ablation of tissue near the ostium of a pulmonary vein.

The responsive tissue stimulator 11 shown in FIG. 2 would include electronic circuitry that is well known in the art of pacemakers, ICDs and responsive neural stimulators such as those described in U.S. Pat. No. 6,016,449. The circuits within the responsive tissue stimulator 11 that would be required include (but are not limited to) amplifiers 20 for the sensing electrodes, analog-to-digital converters 21, digital memories 22, a comparator circuit 23, stimulation pulse generator circuit 24, a command receiver 25 and a telemetry transmitter 26. The use of amplifiers 20 is obvious. The output of the amplifiers 20 would feed into the analog-to-digital converters 21 because modern technology requires most signals to be processed in a digital format. One or more digital memories 22 could be used for several purposes. One purpose would be merely to store the electrical signal from the patient's heart (an electrogram) so that the precursor signal of atrial fibrillation for that particular patient could be determined. After such a precursor signal has been recognized, a template of that signal would be placed into memory. When the patient creates that precursor signal after it has been recorded in memory, the comparator circuitry 23 would be used to determine if such a precursor signal has been created. If the precursor signal is recognized by the comparator circuitry 23, it would provide a signal to one or more electrodes to cause a stimulation pulse train to be impressed upon some tissue of the patient that has been found to decrease episodes of atrial fibrillation. Such tissue may be the vagus nerve(s) in the neck or some tissue of the heart or pulmonary veins that is near the ostium of one or more pulmonary vein. A likely place to stimulate the heart would be in the left atrium. However, other locations within the heart are certainly possible.

Another purpose of the digital memory would be to provide a record of the episodes of atrial fibrillation that have occurred. This record can include a listing of each episode of atrial fibrillation including its start time, end time or time duration and the date and time when that episode occurred. Another type of data that can be recorded is a histogram of the number of episodes of atrial fibrillation on each day and can include the average time duration of these episodes and/or the maximum and minimum time period for the episodes of atrial fibrillation on that particular day. These data can be used by the patient's physician to determine the effectiveness of the treatment for atrial fibrillation and/or to adjust the patient's stimulation parameters to optimize the treatment. The data to be read out would typically be over a set period of time (such as three months) or it can be from the date of the last readout from the digital memory.

The command receiver 25 shown in FIG. 2 is used to receive programming commands from the external equipment 30. such systems are well known for adjusting the operating parameters of pacemakers, ICDs or responsive neural stimulators for the brain. Typical of the functions of the responsive tissue stimulator 11 would be to adjust the voltage amplitude and time duration of the responsive electrical signal. For example, the responsive signal could have a voltage between 0.01 and 15 volts and a time duration from a millisecond to as long as several hours. The long time durations would be applied mostly for vagal nerve stimulation.

The telemetry transmitter 26 would be used to send an rf signal to the external equipment 30 when the patient's attending physician wishes to receive stored or real time data from the implanted responsive tissue stimulator 11. Again, such systems are well known in the field of pacemakers, ICDs and responsive neural stimulators. The signals to or from the responsive tissue stimulator 11 would be by means of the antenna 29 that communicates through the antenna 31 of the external equipment 30.

The implanted responsive tissue stimulator 11 would also include a battery 27 and typically, a d-c to d-c converter 28. The battery 26 could be a primary or a rechargeable battery as is well known in the art of implanted medical devices.

Although certain specific locations for both sensing and stimulation electrodes have been described herein, it should be understood that particular patients may have different regions of their body where either sensing or stimulation is optimally applied for reducing the incidence of atrial fibrillation. These locations include anyplace in the region of the heart and/or any nerve whose stimulation can reduce the incidence of atrial fibrillation.

Various other modifications, adaptations and alternative designs are of course possible in light of the teachings as presented herein. Therefore it should be understood that, while still remaining within the scope and meaning of the appended claims, this invention could be practiced in a manner other than that which is specifically described herein.

What is claimed is:

1. A responsive tissue stimulator system for reducing the incidence of atrial fibrillation in a human subject, the system including;
at least one sensing electrode to sense a precursor of an episode of atrial fibrillation or an early onset electrical signal of the actual atrial fibrillation;
at least one stimulation electrode for providing electrical stimulation of some tissue of that human subject when the sensing electrode senses the atrial fibrillation or its precursor electrical signal, the location of the electrical stimulation being tissue that, when electrically stimulated, causes a reduction in the incidence or severity of atrial fibrillation; and,
electronic circuitry within the implanted responsive tissue stimulator that is electrically connected to both the at least one sensing electrode and the at least one stimulation electrode, the electronic circuitry including a digital memory that is designed to store the atrial fibrillation signal or its precursor signal that originates from the at least one sensing electrode and creating a response electrical stimulation signal that is placed onto the at least one stimulation electrode when atrial fibrillation or its precursor electrical signal is sensed.

2. The responsive tissue stimulator system of claim 1 where the at least one sensing electrode is located near the apex of the right ventricle of the patient's heart.

3. The responsive tissue stimulator system of claim 1 where the at least one stimulation electrode is located in close proximity to a vagal nerve in the patient's neck.

4. The responsive tissue stimulator system of claim 3 where one stimulation electrode is located in close proximity to the left vagal nerve in the neck.

5. The responsive tissue stimulator system of claim 1 where the digital memory also includes data on the time history of the occurrence of episodes of atrial fibrillation.

6. The responsive tissue stimulator system of claim 5 where the data stored indicates the start time, end time or time duration and the date and time for each episode of atrial fibrillation that has occurred since the patient's physician last read out the stored data.

7. The responsive tissue stimulator system of claim 5 where the occurrences of atrial fibrillation are presented in a histogram format that provides the number of episodes of atrial fibrillation on each day since the last readout by the patient's physician.

8. The responsive tissue stimulator system of claim 1 where the time extent of the responsive electrical signal can be programmed to be from a fraction of a second to as long as several hours.
